(19) United States
(12) Patent Application Publication
(10) Pub. No.: US 2022/0091132 A1
(43) Pub. Date: Mar. 24, 2022

(54) CELL-BASED ASSAY FOR DETECTION OF ANTIBODIES IN A SAMPLE

(71) Applicant: Becton, Dickinson and Company, Franklin Lakes, NJ (US)

(72) Inventors: Oleg Guryev, San Jose, CA (US); Majid Mehrpouyan, Gilroy, CA (US)

(21) Appl. No.: 17/395,143

(22) Filed: Aug. 5, 2021

Related U.S. Application Data
(60) Provisional application No. 63/081,647, filed on Sep. 22, 2020.

(51) Int. Cl. G01N 33/68

(52) U.S. Cl. CPC G01N 33/6854; G01N 2333/165; G01N 2469/20

(57) ABSTRACT

Methods of determining whether a sample includes an antibody that binds to a coronaviral antigen are provided. Aspects of the methods may include combining the sample, a mammalian cell displaying on a surface thereof an antigenic determinant of the coronaviral antigen that is specifically bound by the antibody, and a labeled secondary binding member that binds to the antibody to produce an assay composition; and flow cytometrically assaying the assay composition for the presence of a signal from the labeled secondary binding member to determine whether the sample includes the antibody. Also provided are methods of assessing a subject for an immune response to a coronaviral infection and methods of producing a mammalian cell displaying on a surface thereof an antigenic determinant of a coronaviral antigen. Also provided are systems, expression vectors, mammalian cells, and kits for practicing the subject methods.

### CROSS-REFERENCE

Pursuant to 35 U.S.C. § 119 (e), this application claims priority to the filing date of U.S. Provisional Patent Application Ser. No. 63/081,647 filed Sep. 22, 2020; the disclosures of which applications are incorporated herein by reference in their entirety.

### INTRODUCTION

Coronaviruses are enveloped, positive-sense single-stranded RNA viruses. They have the largest genomes (26-32 kb) among known RNA viruses, and are phylogenetically divided into four genera (alpha, beta, gamma, delta), with betacoronaviruses further subdivided into four lineages (A, B, C, D). Coronaviruses infect a wide range of avian and mammalian species, including humans. Of the six known human coronaviruses, four of them (HCoV-OC43, HCoV-229E, HCoV-HKU1 and HCoV-NL63) circulate annually in humans and generally cause mild respiratory diseases, although severity can be greater in infants, elderly, and the immunocompromised. In contrast, the Middle East respiratory syndrome coronavirus (MERS-CoV) and the severe acute respiratory syndrome coronavirus (SARS-CoV), belonging to betacoronavirus lineages C and B, respectively, are highly pathogenic.

In 2019, a novel coronavirus (2019-nCoV/SARS-CoV-2) instigated a major outbreak of respiratory disease, which was originally centered on Hubei province, China. The disease is now a global pandemic which is showing little signs of abatement. Taxonomically, SARS-CoV-2 is a betacoronavirus, which is thought to be of lineage A or C (Jaimes et al., “Phylogenetic Analysis and Structural Modeling of SARS-CoV-2 Spike Protein Reveals an Evolutionary Distinct and Proteolytically Sensitive Activation Loop,” J. Mol. Biol. (May 1, 2020) 432(10): 3309-3325). COVID-19, the disease caused by SARS-CoV-2, may manifest with a number of clinical symptoms, including pneumonia, fever, dry cough, headache, and dyspnea. In some instances, the disease may progress to respiratory failure and death. Id.

A diagnostic test for determining if a patient has COVID-19 is a real time reverse transcription polymerase chain reaction (RT-PCR) test for the qualitative detection of nucleic acid from SARS-CoV-2 in respiratory samples. The test is used to identify SARS-CoV-2 RNA in a patient sample, and a positive test result indicates the patient has an active coronavirus infection. In a typical protocol, a patient or healthcare provider collects a respiratory sample from the nose or throat of the patient using a swab. The swab is placed in a sealed, sterile container and transported to a laboratory within 72 hours. At the laboratory, viral RNA is extracted from the swab and RT-PCR is performed where viral RNA is reverse transcribed to DNA and then amplified using primers specific to regions of the viral genome. The presence of the DNA may then be indicated with probes that provide a fluorescent signal when bound to the DNA. The RT-PCR test may be administered to individual samples including self-collected nasal swab specimens or with pooled samples.

### SUMMARY

The duration and nature of immunity generated in response to SARS-CoV-2 infection is not known. Current strategies for SARS-CoV-2 testing are based on reverse transcription polymerase chain reaction (RT-PCR) and do not provide information about the host immune response to the virus. Furthermore, the RT-PCR based test does not indicate if an asymptomatic patient has antibodies or immunity to SARS-CoV-2.

Methods of determining whether a sample includes an antibody that binds to a coronaviral antigen are provided. Aspects of the methods may include combining the sample, a mammalian cell displaying on a surface thereof an antigenic determinant of the coronaviral antigen that is specifically bound by the antibody, and a labeled secondary binding member that binds to the antibody to produce an assay composition. The resultant assay composition is then flow cytometrically assayed for the presence of a signal from the labeled secondary binding member to determine whether the sample includes the antibody. Also provided are methods of assessing a subject for an immune response to a coronaviral infection and methods of producing a mammalian cell displaying on a surface thereof an antigenic determinant of a coronaviral antigen. Also provided are systems, expression vectors, mammalian cells, and kits for practicing the subject methods.

### BRIEF DESCRIPTION OF THE FIGURES

FIG. 1 provides a schematic representation of an embodiment of a membrane anchored protein.

FIG. 2 provides a schematic representation of an embodiment of an expression vector.

FIG. 3 depicts a flow cytometer according to certain embodiments.

FIG. 4 provides a schematic representation of an embodiment of a cell-based serological assay for antibodies specific to a coronaviral antigen.

### DEFINITIONS

As used herein in its conventional sense, the terms “Ag” or “antigen” refer to a substance capable of either binding to an antigen binding region of an immunoglobulin molecule or of eliciting an immune response. As used herein, “antigen” includes, but is not limited to, antigenic determinants, haptens, and immunogens which may be peptides, small molecules, carbohydrates, lipids, nucleic acids or combinations thereof. The skilled immunologist will recognize that when discussing antigens that are processed for presentation to T cells, the term “antigen” refers to those portions of the antigen (e.g., a peptide fragment) that is a T cell epitope presented by MHC to the T cell receptor. When used in the context of a B cell mediated immune response in the form of an antibody that is specific for an “antigen”, the portion of the antigen that binds to the complementarity determining regions of the variable domains of the antibody (light and heavy) the bound portion may be a linear or three-dimensional epitope.

As used herein, the term “antigenic determinant” or “epitope” as used in its conventional sense refers to a site on an antigen to which an immunoglobulin or antibody (or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes 3, 4, 5, 6, 7, 8, 9, 10, 11, 12, 13, 14, 15 or more amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

As used herein, the term “coronavirus” as used in its conventional sense refers to a virus from the family of envelope, single-stranded, positive-strand RNA viruses classified within the Nidovirales order. The coronaviral genome encodes four major structural proteins: the spike (S) protein, nucleocapsid (N) protein, membrane (M) protein, and the envelope (E) protein, all of which are typically required to produce a structurally complete viral particle. The structure of coronaviruses is further described in Schoeman and Fielding (2019) Virology Journal 16 (29), the disclosure of which is incorporated by reference herein in its entirety.

As used herein, the term “fluorescent protein” refers to any protein capable of fluorescence when excited with appropriate electromagnetic radiation. This includes fluorescent proteins whose amino acid sequences are either naturally occurring or engineered (i.e., analogs or mutants). Many cnidarians use green fluorescent proteins (“GFPs”) as energy-transfer acceptors in bioluminescence. A “green fluorescent protein,” as used herein, is a protein that fluoresces green light. Similarly, “blue fluorescent proteins” fluoresce blue light and “red fluorescent proteins” fluoresce red light. GFPs have been isolated from the Pacific Northwest jellyfish, Aequorea victoria, the sea pansy, Renilla reniformis, and Phialidium gregarium. W. W. Ward et al., Photochem. Photobiol., 35:803-808 (1982); L. D. Levine et al., Comp. Biochem. Physiol., 726:77-85 (1982). Fluorescent proteins and protein variants may also be derived from reef coral such as the Living Colors® fluorescent proteins. The Living Colors® fluorescent proteins are suited for, e.g., whole-cell labeling, promoter-reporter studies, imaging, use as a transfection control, use as a tag or reporter, gene function and expression studies, fusion studies, and optical labeling and tracking live cells and organelles. Exemplary Living Colors® fluorescent proteins include mPlum, E2-Crimson, mRaspberry, HcRed, mCherry, mStrawberry, AsRed, DsRed, tdTomato, mOrange, mBanana, ZsYellow, ZsGreen, AcGFP, Dendra2, Timer, PAmCherry, AmCyan, EYFP, EGFP, ECFP, and variants thereof.

As used herein, the term “fusion polypeptide” or “fusion protein” means a protein created by joining two or more polypeptide sequences together. The fusion polypeptides may include translation products of a chimeric gene construct that joins the DNA sequences encoding one or more antigens, or fragments or mutants thereof, with the DNA sequence encoding a second polypeptide (in some cases, additional DNA sequences encoding a third or fourth polypeptide, etc.) to form a single open-reading frame. A “fusion polypeptide” or “fusion protein” may refer to a recombinant protein of two or more proteins which are joined by a peptide bond.

A used herein, the term “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “immunity” refers to protection from disease (e.g., preventing or attenuating (e.g., suppression) of a sign, symptom or condition of the disease) upon exposure to a microorganism (e.g., pathogen) or virus capable of causing the disease. Immunity can be innate (e.g., non-adaptive (e.g., non-acquired) immune responses that exist in the absence of a previous exposure to an antigen) and/or acquired (e.g., immune responses that are mediated by B and T cells following a previous exposure to antigen (e.g., that exhibit increased specificity and reactivity to the antigen)).

### DETAILED DESCRIPTION

Methods of determining whether a sample includes an antibody that binds to a coronaviral antigen are provided. Aspects of the methods may include combining the sample, a mammalian cell displaying on a surface thereof an antigenic determinant of the coronaviral antigen that is specifically bound by the antibody, and a labeled secondary binding member that binds to the antibody to produce an assay composition. The assay composition is then flow cytometrically assayed for the presence of a signal from the labeled secondary binding member to determine whether the sample includes the antibody. Also provided are methods of assessing a subject for an immune response to a coronaviral infection and methods of producing a mammalian cell displaying on a surface thereof an antigenic determinant of a coronaviral antigen. Also provided are systems, expression vectors, mammalian cells, and kits for practicing the subject methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, aspects of the invention include methods of determining whether a sample includes an antibody that binds to a coronaviral antigen. By “determining the presence of” is meant assaying a sample for a signal associated with a component, e.g., antibody, in the sample, wherein the presence of the signal indicates that the component is present in the sample. The determining may include obtaining the signal by visual or instrumental means. In some cases, the determining includes detecting a signal from a sample, e.g., from a component in the sample, where the signal indicates the component is present in the sample. The signal may be produced by a molecule that itself produces a signal that can be detected such as, e.g., a fluorescent, chemiluminescent or radioactive signal, etc. Alternatively, the signal may be produced by a molecule that requires reaction with another molecule to generate a signal that can be detected.

In practicing embodiments of the methods, the methods may include combining: the sample, a mammalian cell displaying on a surface thereof an antigenic determinant of the coronaviral antigen that is specifically bound by the antibody, and a labeled secondary binding member that binds to the antibody to produce an assay composition; and flow cytometrically assaying the assay composition for the presence of a signal from the labeled secondary binding member to determine whether the sample includes the antibody. The presence or detection of a signal from the labeled secondary binding member may indicate that the sample includes the antibody. The absence of the signal from the labeled secondary binding member may indicate that the sample does not include the antibody.

### Production of Assay Composition

As summarized above, aspects of the subject methods may include producing an assay composition for flow cytometric analysis. The producing may include, e.g., combining a sample, a mammalian cell displaying on a surface thereof an antigenic determinant of the coronaviral antigen that is specifically bound by the antibody, and a labeled secondary binding member that binds to the antibody. The methods may further include producing or preparing each of the components of the assay composition. The components of the assay composition, e.g., the mammalian cell, the labeled secondary binding member, and the sample, are discussed in detail below.

### Mammalian Cell

As summarized above, one component of the assay composition that is produced in embodiments of the methods is a mammalian cell. The mammalian cells employed in embodiments of the invention display on the surface thereof an antigenic determinant of a coronaviral antigen. The mammalian cells may express the antigenic determinant transiently or stably. In some cases, mammalian cells express a membrane anchored protein according to any of the embodiments described herein.

The term “mammalian host cell,” “host cell”, “mammalian cell” and the like, refers to cells of cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in mammalian Cell Culture (Mather, J. P. ed., Plenum Press, N.Y. [1984]), and Barnes and Sato, (1980) Cell, 22:649. The cells can be maintained according to standard methods well known to those of skill in the art (see, e.g., Freshney (1994) Culture of Animal Cells, A Manual of Basic Technique, (3d ed.) Wiley-Liss, New York; Kuchler et al. (1977) Biochemical Methods in Cell Culture and Virology, Kuchler, R. J., Dowden, Hutchinson and Ross, Inc. and the references cited therein).The mammalian host cells can be derived from any suitable species, including but not limited to rat, mouse, bovine, porcine, sheep, goat, and human.

The mammalian cell may be derived from any suitable mammalian cell line. The mammalian cell may be a cell capable of producing proteins with post-translational modifications such as those modifications produced, e.g., in humans. Post-translational modifications may include, e.g., glycosylation, palmitoylation, ADP ribosylation, phosphorylation, acetylation, hydroxylation, methylation, lipidation, etc. The cells further may include the appropriate machinery for properly folding expressed proteins. In some cases, the mammalian cell line is a human cell line. The mammalian cell line may be any mammalian cell line capable of expressing a viral particle, viral proteins, or portions thereof including, e.g., the antigenic determinant of the coronaviral antigen. Examples of suitable mammalian host cells include HeLa cells (HeLa S3 cells, ATCC CCL2.2), Jurkat cells, Raji cells, Daudi cells, human embryonic kidney cells (293-HEK; ATCC 293c18, ATCC CRL 1573), African green monkey kidney cells (CV-1; Vero; ATCC CRL 1587), SV40-transformed monkey kidney cells (COS-1; ATCC CRL 1650), canine kidney cells (MDCK; ATCC CCL 34), baby hamster kidney cells (BHK-21, BHK-570; ATCC CRL 8544, ATCC CRL 10314), Chinese hamster ovary cells (CHO-K1; ATCC CCL61; CHO DG44 (Chasin et al., 1986, Som Cell Molec Genet, 12, 555)), rodent cell lines such as NSO, SP2/O, GH1 (ATCC CCL82), H-4-II-E (ATCC CRL 1548), NIH-3T3 (ATCC CRL 1658), HT-1080 cell line, PER.C6 cell line, HKB-11 cell line, HuH-7 cell line. Additional examples of suitable mammalian cell lines are described, e.g., in U.S. Patent No. 7,666,416 and U.S. Patent Publication No. 2015/0079659, the disclosures of which are incorporated by reference herein in their entireties.

In some cases, the mammalian cell is a HEK293 cell or a derivative thereof. The human embryonic kidney cell line 293 (“293 cells”), which exhibits epithelial morphology, has proven useful, e.g., for studies of the expression of exogenous ligand receptors, production of viruses, and expression of allogeneic and xenogeneic recombinant proteins. 293 cells have been used to produce viruses such as natural and recombinant adenoviruses (Garnier, A., et al., Cytotechnol. 15:145-155 (1994); Bout, A., et al., Cancer Gene Therapy 3(6):524, abs. P-52 (1996); Wang, J.-W., et al., Cancer Gene Therapy 3(6):524, abs. P-53 (1996)), which may be used, e.g., for vaccine production or construction of viral vectors for recombinant protein expression. 293 cells have also proven useful in large-scale production of a variety of recombinant human proteins (Berg, D. T., et al., Bio Techniques 14(6):972-978 (1993); Peshwa, M. V., et al., Biotechnol. Bioeng. 41:179-187 (1993); Garnier, A., et al., Cytotechnol. 15:145-155 (1994)). Variant HEK293 cell lines include, e.g., HEK293F, HEK293FT, HEK293T, HEK293S, HEK293FTM, HEK293SG, HEK293SGGD, HEK293H, HEK293E, HEK293EBNA1-6E, HEK293MSR, and HEK293A.

In some cases, the mammalian cell is a CHO cell. CHO cells have been classified as both epithelial and fibroblast cells derived from the Chinese hamster ovary. The CHO cell can be a derivative of a CHO-K1 cell, a derivative of a CHO-DUXB11 cell, a CHO-DG44 cell, a CHO-SSF3 cell or a derivative of a CHO-S cell.

As discussed above, the mammalian cell may express an antigenic determinant of a coronaviral antigen. The antigenic determinant of the coronaviral antigen may be derived from any suitable coronavirus. In some cases, the coronaviral antigen is derived from a SARS-CoV. In some cases, the coronaviral antigen is derived from a MERS-CoV. In some cases, the coronaviral antigen is derived from a SARS-CoV-2. The coronaviral antigen may be any protein or fragment thereof that facilitates infection by the virus. In certain embodiments, the antigenic determinant includes a coronaviral structural protein or a fragment thereof. Where the antigenic determinant includes a fragment of a coronaviral structural protein, the fragment may vary in size such that the size of the fragment ranges from, e.g., 3 to 30 amino acids, 5 to 30 amino acids, 8 to 30 amino acids, 10 to 30 amino acids, 5 to 20 amino acids, 5 to 10 amino acids, 4 to 6 amino acids, 3 to 5 amino acids, or 4 to 5 amino acids. In certain embodiments, the antigenic determinant includes a binding site for an antibody or fragment thereof on a coronaviral protein, e.g., structural protein. In some cases, the antigenic determinant includes an epitope that is conserved between one or more coronaviruses.

In some cases, the coronaviral antigen includes a spike protein or a fragment thereof. In some cases, the antigenic determinant includes the receptor binding domain or fragment thereof, e.g., in the S protein of a coronavirus. In some cases, the antigenic determinant includes the N-terminal domain or fragment thereof, e.g., in the S protein of a coronavirus. The MERS-CoV S protein and/or exemplary antigenic determinants of interest on a MERS-CoV spike protein are described in, e.g., U.S. Pat. Nos. 10,421,802; 10,406,222; 10,301,377; 10,131,704; 9,718,872; U.S. Publication No.'s: 20190351049; 20190256579; 20190194299; PCT Publication No.'s: 2015057942; 2015164865A1; 2015179535A1, the disclosures of which are incorporated by reference herein in their entireties. The SARS-CoV S protein and/or exemplary antigenic determinants of interest on a SARS-CoV spike protein are described in U.S. Pat. Nos. 8,106,170; 7,897,744; 7,750,123; 7,728,110; 7,629,443; 7,622,112; 7,396,914; U.S. Publication No.'s 20120082693; 20110178269; 20110159001; 20090304683; 20080286756; 20080069830; 20080027006; 20070128217; 20070116716; 20080269115, the disclosures of which are incorporated by reference herein in their entireties. The SARS-CoV-2 S protein and/or exemplary antigenic determinants of interest on a SARS-CoV-2 spike protein are described in Zhang et al. (2009) Viral Immunol. 22(6): 407-15; Yuan et al. (2020) Science 368(6491): 630-633; Pinto et al. (2020) Nature 583: 290-295; Tian et al. (2020) Emerging Microbes and Infections. 9: 382-385; Chi et al. (2020) Science 369(6504): 650-655; Yi et al. (2020) Cellular & Molecular Immunology 17: 621-630; Ravichandran et al. (2020) Science Translational Medicine 12(550): eabc3539, the disclosures of which are incorporated by reference herein in their entireties.

In some cases, the coronaviral antigen includes an envelope protein or a fragment thereof. The MERS-CoV envelope protein and/or exemplary antigenic determinants of interest on a MERS-CoV envelope protein are described in Surya et al. (2015) Virus Res. 201:61-6; Schoeman et al. (2019) Virol J. 16:69, the disclosures of which are incorporated herein by reference in their entireties. The SARS-CoV envelope protein and/or exemplary antigenic determinants of interest on a SARS-CoV envelope protein are described in U.S. Pat. No. 7,897,744; U.S. Publication No.'s 20080269115; 20070128217, the disclosures of which are incorporated by reference herein in their entireties. The SARS-CoV-2 envelope protein and/or exemplary antigenic determinants of interest on a SARS-CoV-2 envelope protein are described in Bianchi et al. (2020) BioMed Research International 2020: 1-6; Tilocca et al. (2020) Microbes Infect. 22(4): 182-187, the disclosures of which are incorporated by reference herein in their entireties.

In some cases, the coronaviral antigen includes a membrane protein or a fragment thereof. The MERS-CoV membrane protein and/or exemplary antigenic determinants of interest on a MERS-CoV membrane protein are described in Perrier et al. (2019) J Biol Chem. 294(39): 14406-14421; Alsaadi et al. (2019) Future Virol. 14(4): 275-286, the disclosures of which are incorporated by reference herein in their entireties. The SARS-CoV membrane protein and/or exemplary antigenic determinants of interest on a SARS-CoV membrane protein are described in U.S. Pat. No. 7,897,744; U.S. Publication No.'s 20080269115; 20070128217; Liu et al. (2010) The Journal of Infectious Diseases 202(8): 1171-1180, the disclosures of which are incorporated by reference herein in their entireties. The SARS-CoV-2 membrane protein and/or exemplary antigenic determinants of interest on a SARS-CoV-2 membrane protein are described in Bianchi et al. (2020) BioMed Research International 2020: 1-6; Tseng et al. (2013) PLoS One 8(5): e64013, the disclosures of which are incorporated herein by reference in their entireties.

In some cases, the coronaviral antigen includes a nucleocapsid protein or a fragment thereof. The MERS-CoV nucleocapsid protein and/or exemplary antigenic determinants of interest on a MERS-CoV nucleocapsid protein are described in PCT Publication No. 2016116398, the disclosure of which is incorporated herein by reference in its entirety. The SARS-CoV nucleocapsid protein and/or exemplary antigenic determinants of interest on a SARS-CoV nucleocapsid protein are described in U.S. Pat. Nos. 7,696,330; 7,897,744; 7,696,330; 8,343,718; U.S. Publication No.'s: 20080269115; 20100172917; 20090280507; 20080254440; 20070128217, the disclosures of which are incorporated by reference herein in their entireties. The SARS-CoV-2 nucleocapsid protein and/or exemplary antigenic determinants of interest on a SARS-CoV-2 nucleocapsid protein are described in Dutta et la. (2020) Journal of Virology 94(13): e00647-20; Zeng et al. (2020) Biochem Biophys Res Commun. 527(3): 618-623; Kang et al. (2020) Acta Pharmaceutica Sinica B 10(7):1228-1238, the disclosures of which are incorporated herein by reference in their entireties.

Antigenic determinants on SARS-CoV proteins are further discussed in, e.g., Liang et al. (2005) Clin Chem. 51(8): 1382-1396; Shih et al. (2006) Journal of Virology 80(21): 10315-10324; Brink et al. (2005) J Virol. 79(3): 1635-1644; Zhang et al. (2009) Viral Immunology 22(6): 407, the disclosures of which are incorporated herein by reference in their entireties. Antigenic determinants on MERS-CoV proteins are further discussed in, e.g., Widjaja et al. (2019) Emerg Microbes Infect. 8(1): 516-530; Yu et al. (2015) Sci Rep. 5: 13133; Zhou et al. (2019) Nature Communications 10; Tai et al. (2017) Journal of Virology 91(1): e01651-16; Wang et al. (2018) Journal of Virology 92(10): e02002-17; Pallesen et al. (2017) PNAS 114 (35): E7348-E7357; Wang et al. (2019) Cell Reports 28: 3395-3405; Zhang et al. (2018) Cell Rep. 24(2): 441-452, the disclosures of which are incorporated herein by reference in theft entireties.

The antigenic determinant may be displayed by a mammalian cell on the surface of the cell. The antigenic determinant may be displayed on the cell surface, e.g., on the surface outside the cell, by any convenient means. In some cases, an antigenic determinant is fused to a protein subunit that binds to another subunit, wherein the bound subunits are displayed on the surface of the cell, similar to the yeast cell display systems as described in U.S. Pat. Nos. 6,699,658; 6,696,251; and 10,041,064, the disclosures of which are incorporated herein by reference in their entireties. In some cases, the antigenic determinant is a component of an engineered protein that is expressed in a host cell under conditions that result in the protein being modified in such a way that the modified protein binds to a surface-immobilized binding partner if it is secreted from the host cell, e.g., as described in U.S. Pat. No. 9,645,146, the disclosure of which is incorporated herein by reference. In some cases, the antigenic determinant is expressed by a mammalian cell and captured by a capture moiety expressed on the surface of the cell as described in, e.g., U.S. Pat. No. 9,260,712, incorporated herein by reference in its entirety. In certain embodiments, the antigenic determinant is encoded in a vector, e.g., a viral vector, that is introduced into a mammalian cell which may be cultured to allow expression and cell surface presentation of the vector encoded antigenic determinant as described in, e.g., U.S. Pat. No. 9,567,389, incorporated herein by reference in its entirety. Mammalian cell surface display vectors are further described in, e.g., U.S. Pat. Nos. 8,163,546 and 9,134,309, incorporated herein by reference in their entireties.

In certain embodiments, the antigenic determinant expressed by the mammalian cell may be a component of a membrane anchored protein. The membrane anchored protein may be a fusion protein including one or more joined polypeptides. In some cases, the membrane anchored protein includes the following joined domains: an antigenic determinant of a coronaviral antigen, e.g., as described above, a membrane anchor domain, and a detectible domain. The membrane anchored protein or portion thereof may be embedded in the cell membrane (phospholipid bilayer of a cell) by any suitable means, as described below. The membrane anchored protein may be embedded in the cell membrane in an orientation where the antigenic determinant is positioned on the surface of the cell, e.g., outside the cell membrane and opposite to the cytoplasmic side of the cell membrane. The membrane anchored protein may be embedded in the cell membrane in an orientation where the detectible domain is positioned inside the cell, e.g., on the cytoplasmic side of the cell membrane.

The membrane anchor protein may include a membrane anchor domain. The membrane anchor domain may anchor the membrane anchored protein to a cell membrane. By “anchor” is meant permanently or transiently stably associating or coupling the protein with the cell membrane, e.g., permanently or transiently binding or embedding the protein to the cell membrane. In some cases, the membrane anchor domain includes hydrophobic residues, e.g., residues with hydrophobic side chains, that interact with the fatty acyl groups of membrane phospholipids, thereby embedding the membrane anchored domain into the phospholipid bilayer. In some cases, the membrane anchored protein, e.g., the membrane anchor domain, includes an integral membrane protein. In some cases, the membrane anchored protein, e.g., the membrane anchor domain, includes a transmembrane protein. In some cases, the membrane anchored protein, e.g., the membrane anchor domain, includes a lipid anchor. In such embodiments, a portion of the membrane anchored protein may be covalently bound to one or more fatty acids or hydrocarbon chains that anchor the protein to the membrane. In some embodiments, an expression construct encoding the membrane anchored protein may encode a signal for a lipid anchor addition. Suitable lipid anchors include, e.g., glycosylphosphatidylinositol (GPI) anchors, fatty acids (e.g., myristic acid, palmitic acid, etc.), isoprenyl groups (e.g., farnesyl and geranylgeranyl), sterols (e.g., cholesterol), and phospholipids. Exemplary lipid anchors are described in (Triffo et al. (2012) J. Am. Chem. Soc. 134: 10833-10842) and Nalivaeva N. N., Turner A. J. (2009) Lipid Anchors to Proteins. In: Lajtha A., Tettamanti G., Goracci G. (eds) Handbook of Neurochemistry and Molecular Neurobiology. Springer, Boston, MA, the disclosures of which are incorporated herein by reference in their entireties.

In some cases, the membrane anchored protein includes a detectible domain. The detectible domain may provide a detectible signal which, e.g., may indicate that the membrane anchored protein has been expressed by the cell. In some cases, the detectible domain allows the cell expressing the membrane anchored protein to be detected by instrumental means, e.g., flow cytometrically. In some cases, the membrane anchored protein is embedded in the cell membrane in an orientation where the detectible domain is positioned or present inside the cell. In some cases, the detectible domain includes a fluorescent protein. Fluorescent proteins that can be used include, e.g., biological fluorophores. Exemplary biological fluorophores include T-sapphire, Cerulean, mCFPm, CyPet, EGFP, PA-EGFP, Emerald, EYFP, Venus, mCitrine, mKO, mOrange, DSRed, JRed, mStrawberry, mCherry, PA-mCherry, mRuby, Tomato, mPlum, mKate, mKatushka, Kaede, Halotag, and superecliptic fluorine. Suitable fluorescent proteins are further described in, e.g., U.S. Pat. Nos. 8,999,669; 7,157,566; 7,560,287; 10,753,846 and U.S. Publication No.'s 20150010917; 20170073671; 20190233482, the disclosures of which are incorporated herein by reference in their entireties.

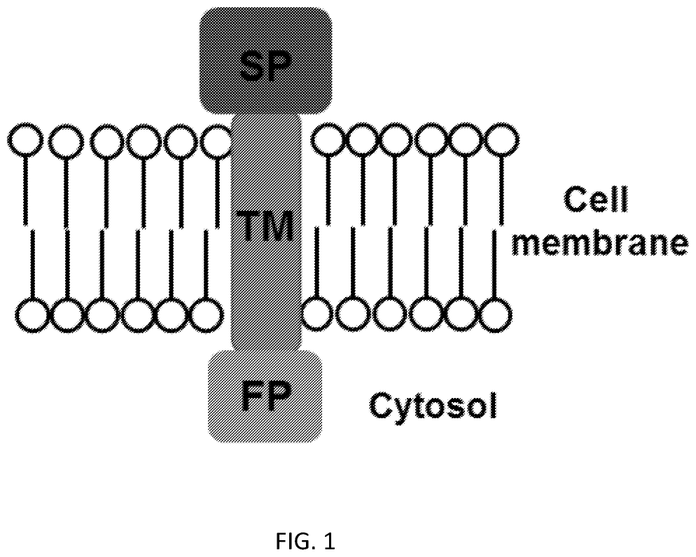

FIG. 1 provides an embodiment of a membrane anchored protein. FIG. 1 shows a schematic representation of a fusion protein expressed in an HEK-293 cell plasma membrane. The fusion protein includes the following components: a SARS-CoV-2 protein (SP), a transmembrane domain (TM), and a green fluorescent protein (FP). The transmembrane domain of the fusion protein spans the cell membrane and attaches the fusion protein to the cell membrane. The fusion protein is oriented such that the SARS-CoV-2 protein is present outside the cell or at the exterior of the cell membrane. In such an orientation, the green fluorescent protein is located inside the cell or on the cytosolic side of the cell membrane.

Aspects of the methods may further include producing a mammalian cell displaying on a surface thereof an antigenic determinant of a coronaviral antigen. The methods may include introducing, e.g., transferring, into a mammalian cell an expression construct encoding a membrane anchored protein including the antigenic determinant of the coronaviral antigen; and maintaining the cell under conditions sufficient for the cell to express the membrane anchored protein. The introducing may include transporting an expression vector, e.g., a nucleic acid construct, from a location outside of a cell to a location inside of a cell, such that the expression vector is moved from a first location on one side of cell membrane to a second location on the other side of a cell membrane. As such, aspects of the methods include methods of moving an expression vector into a target cell, e.g., a mammalian cell, from the location outside of the target cell.

The introducing may occur by any convenient protocol. In some cases, the introducing includes genetically modifying the mammalian cell to include the expression vector. A cell has been “genetically modified” or “transformed” or “transfected” by exogenous DNA or exogenous RNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that include a population of daughter cells containing the transforming DNA. A “clone” is a population of cells derived from a single cell or common ancestor by mitosis. A “cell line” is a clone of a primary cell that is capable of stable growth in vitro for many generations. In some cases, the expression construct is genomically integrated. In some cases, the expression construct is episomal. Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like. In some cases, the genome of a cell is edited by a complex including a guide RNA and a CRISPR effector protein, e.g., Cas9, used for targeted cleavage of a nucleic acid.

In some cases, the introducing includes transfecting a mammalian cell with an expression vector. Aspects of the methods may include producing a transfection composition. The transfection composition may include one or more expression vectors, e.g., as described above, to be introduced into the cell and, e.g., a transfection agent which may include, in some instances, a cationic lipid, peptides, dendrimers, viral vectors, etc. The transfection composition may be a liquid composition. The amount of an expression vector present in the transfection composition may vary, ranging in some instances from 1 ng to 500 μg, such as 10 ng to 100 μg and including 150 ng to 50 μg. The amount of transfection agent present in the liquid transfection composition may vary, ranging in some instances from 1 ng to 5,000 μg, such as 2 ng to 2,500 μg and including 4 ng to 1,400 μg. The transfection composition may be produced using any convenient protocol.

Following production of the transfection composition, the resultant transfection composition may be contacted with the mammalian cell under conditions sufficient for the expression vector to be introduced into the mammalian cell. In these embodiments, the mammalian cell may be contacted with the transfection composition under transfection conditions. In certain instances, an amount of transfection composition is contacted with an amount of mammalian cells under cell culture conditions and incubated for a sufficient period of time for transfection to occur. In some instances, an amount of transfection composition that provides for an amount of an expression vector, e.g., a nucleic acid, ranging from 1 molecule per cell to 1×10E¹² molecules per cell, such as 7×10E⁷ molecules per cell to 7×10E⁸ molecules per cell is contacted with an amount of mammalian cells ranging from 1 to 50 million, such as 10,000 to 100,000 cells. Any convenient culture medium may be employed, where culture media of interest include, but are not limited to: RPMI, DMEM, OptiMEM and the like. The cells and transfection composition may be incubated for varying amounts of time, e.g., from 5 minutes to 5 days, such as 1 to 4 hours, at various temperatures, e.g., ranging from 4 to 42, such as 30 to 37° C.

The expression construct may be a nucleic acid construct. The term “nucleic acid construct”, as used herein, refers to an isolated or artificially generated construct including a nucleic acid molecule. Non-limiting examples of nucleic acid constructs are plasmids, cosmids, bacterial artificial chromosomes, and nucleic acid vectors. The term “vector” as used in its conventional sense refers to any vehicle useful to transfer a nucleic acid into a cell. Examples of vectors are plasmid vectors, gene targeting vectors, and viral vectors, for example parvoviral vectors, such as AAV vectors. A vector may include a nucleic acid construct in single-stranded or double-stranded form, and may include additional molecules, for example, DNA-associated proteins or viral capsid or envelope proteins. Vectors for eukaryotic and prokaryotic cells include, for example, linear and circular DNA or RNA, viral vectors, (e.g., retroviral and parvoviral vectors, such as lentivirus-derived, Moloney murine leukemia virus-derived, adenovirus-derived, and AAV-derived vectors). Viral vectors include, but are not limited to: retroviral, e.g., lentiviral, vectors; adenoviral vectors; adeno-associated virus (AAV) viral vectors, feline immunodeficiency virus (FIV) vectors, rabies virus vectors, avian sarcoma leukosis virus (ASLV) vectors, etc.

The nucleic acid construct or nucleic acid that may be introduced into the mammalian cell may vary greatly. Nucleic acids of interest include polymers of any length composed of deoxyribonucleotides, ribonucleotides, or combinations thereof, where the length of the nucleic acids may vary, e.g., 10 bases or longer, 20 bases or longer, 50 bases or longer, 100 bases or longer, 500 bases or longer, 1000 bases or longer, 2000 bases or longer, 3000 bases or longer, 4000 bases or longer, 5000 bases or longer or more bases, where an upper limit in certain embodiments is 150,000 bases or less, such as 50,000 bases or less. In certain aspects, the nucleic acid is a polymer composed of deoxyribonucleotides or ribonucleotides or combinations thereof, ranging in length from 10 to 150,000, such as 1,000 to 50,000 and including 2,000 to 15,000 bases.

Nucleic acids of interest include deoxyribonucleic acids (DNA), such as but not limited to: genomic DNA or fragments thereof, complementary DNA (or “cDNA”, synthesized from any RNA or DNA of interest), recombinant DNA (e.g., plasmid DNA), etc. Nucleic acids of interest also include ribonucleic acids (RNA), which may be any type of RNA (or sub-type thereof) including, but not limited to, a messenger RNA (mRNA), a microRNA (miRNA), a small interfering RNA (siRNA), a transacting small interfering RNA (ta-siRNA), a natural small interfering RNA (nat-siRNA), a ribosomal RNA (rRNA), a transfer RNA (tRNA), a small nucleolar RNA (snoRNA), a small nuclear RNA (snRNA), a long non-coding RNA (IncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared siRNA (esiRNA), a small temporal RNA (stRNA), a signal recognition RNA, a telomere RNA, a ribozyme, or any combination of RNA types thereof or subtypes thereof.

The expression construct may include an expression cassette. The term “expression cassette”, as used herein, refers to a nucleic acid construct including nucleic acid elements sufficient for the expression of a gene product. The expression vector may include a regulatory sequence operably linked to a coding sequence. Regulatory sequences can include promoters (attached either at the 5′ end of the sense strand or at the 3′ end of the antisense strand), enhancers, terminators, operators, repressors, and inducers. Typically, an expression cassette includes a nucleic acid encoding a gene product operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In some embodiments, the promoter is a heterologous promoter. The term “heterologous promoter”, as used herein, refers to a promoter that is not found to be operatively linked to a given encoding sequence in nature. In some embodiments, an expression cassette may include additional elements, for example, an intron, an enhancer, a polyadenylation site, a woodchuck response element (WRE), and/or other elements known to affect expression levels of the encoding sequence.

In some instances, the expression cassette includes a coding sequence that encodes an antigenic determinant of a coronaviral antigen. Expression cassettes of interest in some embodiments may encode one or more antigenic determinants. In some instances, the expression cassette is present on a vector, e.g., a plasmid, an artificial chromosome, e.g. BAC, etc. While the length of the expression cassette and vector on which it is present may vary, in some instances the length ranges from 0.1 kb to 150 kb, such as 1 to 10 kb and including 5 to 10 kb.

The coding sequence of the expression cassette may be exogenous or endogenous. An “exogenous” coding sequence is a nucleotide sequence which is introduced into the host cell, e.g., into the nucleus of the host cell, into the genome of the host cell, etc. The exogenous coding sequence may not be present elsewhere in the genome of the host (e.g., a foreign nucleotide sequence), may be an additional copy of a sequence which is present within the genome of the host but which is integrated at a different site in the genome, or may be a variant (e.g., containing a mutation, e.g., an insertion, a deletion, a substitution, etc.; encoding a fusion protein; and the like) of a sequence which is present within the genome of the host. An “endogenous” coding sequence is a nucleotide sequence which is present within the genome of the host. An endogenous gene can be operatively linked to an operator sequence(s) to produce an expression cassette of the invention by homologous recombination between an operator sequence recombination vector and sequences of the endogenous gene, such that the native promoter is replaced with the regulatory protein responsive element and the endogenous gene becomes part of an inducible expression cassette.

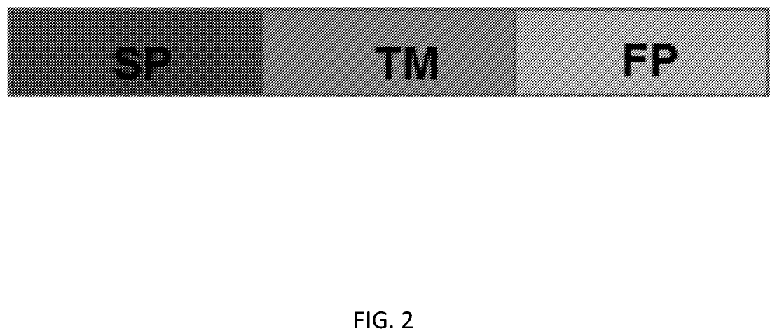

FIG. 2 provides an embodiment of an expression vector. The expression vector in FIG. 2 is a SARS-CoV-2 protein plasmid for use in transfecting HEK-293 cells. The plasmid includes a nucleic acid sequence including a first sequence that encodes a SARS-CoV-2 protein (SP), a second sequence that encodes a transmembrane domain (TM), and a third sequence that encodes a green fluorescent protein (FP).

### Labeled Secondary Binding Member

As summarized above, assay compositions employed in embodiments of the invention also include a labeled secondary binding member that binds to a target antibody that binds to the antigenic determinant of the coronaviral antigen. As used herein, the term “target antibody” refers to the antibody of interest, the presence of which in the sample is to be determined by the subject methods. The target antibody may bind to the antigenic determinant of the coronaviral antigen. In some cases, a result of the subject methods where a signal is detected from the labeled secondary binding member indicates that the target antibody is present in the sample. In some cases, a result of the subject methods where a signal from the labeled secondary binding member is not detected indicates that the target antibody is not present in the sample.

The labeled secondary binding member may be a specific binding member. As used herein, the terms “specific binding member” or “analyte-specific binding member” refer to one member of a pair of molecules that have binding specificity for one another. One member of the pair of molecules may have an area on its surface, or a cavity, which specifically binds to an area on the surface of, or a cavity in, the other member of the pair of molecules. Thus, the members of the pair have the property of binding specifically to each other to produce a binding complex. In some embodiments, the affinity between specific binding members in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁹ M or less, 10⁻¹¹ M or less, 10⁻¹² M or less, 10⁻¹³ M or less, 10⁻¹⁴ M or less, including 10⁻¹⁶ M or less. In some embodiments, the specific binding members specifically bind with high avidity. By high avidity is meant that the binding member specifically binds with an apparent affinity characterized by an apparent K_{d} of 10×10⁻⁹ M or less, such as 1×10⁻⁹ M or less, 3×10⁻¹⁹ M or less, 1×10⁻¹⁹ M or less, 3×10⁻¹¹ M or less, 1×10⁻¹¹ M or less, 3×10⁻¹² M or less or 1×10⁻¹² M or less.

The specific binding member can be proteinaceous. As used herein, the term “proteinaceous” refers to a moiety that is composed of amino acid residues. A proteinaceous moiety can be a polypeptide. In certain cases, the proteinaceous specific binding member is an antibody. In certain embodiments, the proteinaceous specific binding member is an antibody fragment, e.g., a binding fragment of an antibody that specifically binds to an analyte.

As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (k), lambda (I), and heavy chain genetic loci, which together include the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. The term antibody is meant to include full-length antibodies and may refer to a natural antibody from any organism, an engineered antibody, or an antibody generated recombinantly for experimental, therapeutic, or other purposes as further defined below.

In some cases, the specific binding member is an antibody-binding agent. Antibody-binding agents and antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions. In certain embodiments, the specific binding member is a Fab fragment, a F(ab′)2 fragment, a scFv, a diabody or a triabody. In certain embodiments, the specific binding member is an antibody. In some cases, the specific binding member is a murine antibody or binding fragment thereof. In certain instances, the specific binding member is a recombinant antibody or binding fragment thereof.

In some cases, the labeled secondary binding member binds to an FC region of an antibody, e.g., a target antibody that binds to the antigenic determinant of the coronaviral antigen. The term “Fc region” as used herein in its conventional sense refers to the C-terminal region of an immunoglobulin heavy chain. An “Fc region” can be a native sequence Fc region or variant Fc region. In some cases, Fc refers to the last two constant region immunoglobulin domains of IgA, IgD and IgG, the last three constant region immunoglobulin domains of IgE and IgM, and the flexible hinge at the N-terminus of these domains. In the case of IgA and IgM, Fc may include the J chain. In the case of IgG, Fc may include the lower hinge region between the immunoglobulin domains Cgamma2 and Cgamma3 (Cγ2 and Cγ3) and Cgamma1 (Cγ1) and Cgamma2 (Cγ2). Although the boundaries of the Fc region may vary, the human IgG heavy chain Fc region is generally defined as including residues C226 or P230 at its carboxy terminus, where the numbering is according to the EU index as set forth in Kabat. As used herein, “Fc polypeptide” means a polypeptide comprising part or all of the Fc region. Fc polypeptides include antibodies, Fc fusions, isolated Fc and Fc fragments.

In some instances, the specific binding member may be further labeled with a detectable label. In certain embodiments, the specific binding member is an antibody binding agent having a detectable label, e.g., an antibody labeled with fluorescent label. Labels of interest include both directly and indirectly detectable labels. Suitable labels for use in the methods described herein include any molecule that is indirectly or directly detectable by spectroscopic, photochemical, biochemical, immunochemical, electrical, optical, chemical, or other means. Labels of interest include, but are not limited to, fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Labels of interest also include fluorophores, such as indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like.

In some instances, the detectible label is a polymeric dye. Polymeric dyes of interest include, but are not limited to, those dyes described by Gaylord et al. in U.S. Publication Nos. 20040142344, 20080293164, 20080064042, 20100136702, 20110256549, 20110257374, 20120028828, 20120252986, 20130190193, 20160264737, 20160266131, 20180231530, 20180009990, 20180009989, and 20180163054, the disclosures of which are herein incorporated by reference in their entirety; and Gaylord et al., J. Am. Chem. Soc., 2001, 123 (26), pp 6417-6418; Feng et al., Chem. Soc. Rev., 2010,39, 2411-2419; and Traina et al., J. Am. Chem. Soc., 2011, 133 (32), pp 12600-12607, the disclosures of which are herein incorporated by reference in their entirety. The polymeric dye may have one or more desirable spectroscopic properties, such as a particular absorption maximum wavelength, a particular emission maximum wavelength, extinction coefficient, quantum yield, and the like (see e.g., Chattopadhyay et al., “Brilliant violet fluorophores: A new class of ultrabright fluorescent compounds for immunofluorescence experiments.” Cytometry Part A, 81A(6), 456-466, 2012). In some embodiments, the polymeric dye has an absorption curve between 280 nm and 475 nm. In certain embodiments, the polymeric dye has an absorption maximum (excitation maximum) in the range 280 nm and 475 nm. In some embodiments, the polymeric dye absorbs incident light having a wavelength in the range between 280 nm and 475 nm. In some embodiments, the polymeric dye has an emission maximum wavelength ranging from 400 nm to 850 nm, such as 415 nm to 800 nm, where specific examples of emission maxima of interest include, but are not limited to: 421 nm, 510 nm, 570 nm, 602 nm, 650 nm, 711 nm and 786 nm. In some instances, the polymeric dye has an emission maximum wavelength in a range selected from the group consisting of 410 nm to 430 nm, 500 nm to 520 nm, 560 nm to 580 nm, 590 nm to 610 nm, 640 nm to 660 nm, 700 nm to 720 nm, and 775 nm to 795 nm. In certain embodiments, the polymeric dye has an emission maximum wavelength of 421 nm. In some instances, the polymeric dye has an emission maximum wavelength of 510 nm. In some cases, the polymeric dye has an emission maximum wavelength of 570 nm. In certain embodiments, the polymeric dye has an emission maximum wavelength of 602 nm. In some instances, the polymeric dye has an emission maximum wavelength of 650 nm. In certain cases, the polymeric dye has an emission maximum wavelength of 711 nm. In some embodiments, the polymeric dye has an emission maximum wavelength of 786 nm. In certain instances, the polymeric dye has an emission maximum wavelength of 421 nm±5 nm. In some embodiments, the polymeric dye has an emission maximum wavelength of 510 nm±5 nm. In certain instances, the polymeric dye has an emission maximum wavelength of 570 nm±5 nm. In some instances, the polymeric dye has an emission maximum wavelength of 602 nm±5 nm. In some embodiments, the polymeric dye has an emission maximum wavelength of 650 nm±5 nm. In certain instances, the polymeric dye has an emission maximum wavelength of 711 nm±5 nm. In some cases, the polymeric dye has an emission maximum wavelength of 786 nm±5 nm. In certain embodiments, the polymeric dye has an emission maximum selected from the group consisting of 421 nm, 510 nm, 570 nm, 602 nm, 650 nm, 711 nm and 786 nm. Specific polymeric dyes that may be employed include, but are not limited to, BD Horizon Brilliant Dyes, such as BD Horizon Brilliant™ Violet Dyes (e.g., BV421, BV510, BV605, BV650, BV711, BV786); BD Horizon Brilliant™ Ultraviolet Dyes (e.g., BUV395, BUV496, BUV737, BUV805); and BD Horizon Brilliant™ Blue Dyes (e.g., BB515).

Fluorescent labels can be detected using a photodetector (e.g., in a flow cytometer) to detect emitted light. Enzymatic labels are typically detected by providing the enzyme with a substrate and detecting the reaction product produced by the action of the enzyme on the substrate, colorimetric labels can be detected by simply visualizing the colored label, and antigenic labels can be detected by providing an antibody (or a binding fragment thereof) that specifically binds to the antigenic label. An antibody that specifically binds to an antigenic label can be directly or indirectly detectable. For example, the antibody can be conjugated to a label moiety (e.g., a fluorophore) that provides the signal (e.g., fluorescence); the antibody can be conjugated to an enzyme (e.g., peroxidase, alkaline phosphatase, etc.) that produces a detectable product (e.g., fluorescent product) when provided with an appropriate substrate (e.g., fluorescent-tyramide, FastRed, etc.); etc.

### Sample

As summarized above, another component of the assay composition may be a sample. The sample may be a biological sample obtained from a subject. The term “biological sample” is used in its conventional sense to refer to a whole organism, plant, fungi or a subset of animal tissues, cells or component parts which may in certain instances be found in blood, mucus, lymphatic fluid, synovial fluid, cerebrospinal fluid, saliva, bronchoalveolar lavage, amniotic fluid, amniotic cord blood, urine, vaginal fluid and semen. As such, a “biological sample” refers to both the native organism or a subset of its tissues as well as to a homogenate, lysate or extract prepared from the organism or a subset of its tissues, including but not limited to, for example, plasma, serum, spinal fluid, lymph fluid, sections of the skin, respiratory, gastrointestinal, cardiovascular, and genitourinary tracts, tears, saliva, milk, blood cells, tumors, organs. Biological samples may be any type of organismic tissue, including both healthy and diseased tissue (e.g., cancerous, malignant, necrotic, etc.). In certain embodiments, the biological sample is a liquid sample, such as blood or derivative thereof, e.g., plasma, tears, urine, semen, etc., where in some instances the sample is a blood sample, including whole blood, such as blood obtained from venipuncture or fingerstick (where the blood may or may not be combined with any reagents prior to assay, such as preservatives, anticoagulants, etc.). In some cases, the biological sample may be derived from specific biological fluids, such as, but not limited to, blood, plasma, serum, mucus, lymphatic fluid, synovial fluid, cerebrospinal fluid, saliva, bronchoalveolar lavage, amniotic fluid, amniotic cord blood, urine, vaginal fluid and semen.

In certain embodiments the source of the sample is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

In certain embodiments, the sample includes an antibody to a coronaviral antigen, e.g., an antibody that binds to an antigenic determinant of the coronaviral antigen. The antibody may be present in the sample if the subject from which the sample was obtained was exposed to a coronavirus and has had an immune response to the coronavirus. In some cases, the antibody that binds to the coronaviral antigen is an IgM antibody. In some cases, the antibody that binds to the coronaviral antigen is an IgG antibody. In some cases, the antibody that binds to the coronaviral antigen is an IgA antibody.

### Assay Composition Preparation

Combination of the above elements may be achieved using any convenient protocol, including stirring, agitation, etc. The combining of each of the elements, e.g., the sample, the mammalian cell, and the labeled secondary binding member, may occur simultaneously or sequentially. In certain embodiments, the combining may include contacting the sample with a mammalian cell displaying on a surface thereof an antigenic determinant of the coronaviral antigen that is specifically bound by the antibody and a labeled secondary binding member that binds to the antibody to produce an assay composition. In certain embodiments, the combining includes incubating the sample with the mammalian cell and the labeled secondary binding member. In certain embodiments, the combining includes mixing each of the elements, e.g., the sample, the mammalian cell, and the labeled secondary binding member. The mixing may be performed using an agitator. The agitator may be any convenient agitator sufficient for mixing the liquid inside a liquid container, including, but not limited to, vortexers, sonicators, shakers (e.g., manual, mechanical, or electrically powered shakers), rockers, oscillating plates, magnetic stirrers, static mixers, rotators, blenders, mixers, tumblers, orbital shakers, among other agitating protocols. The combination may occur at a variety of temperatures, where the temperature ranges in some instances from 16 to 30° C., such as 20 to 25° C. The combining may occur for any suitable amount of time where the amount of time ranges in some instances from 1 minute to 30 minutes, from 30 minutes to 1 hour, from 30 minutes to 2 hours, from 30 minutes to 5 hours, from 30 minutes to 8 hours, from 1 day to 2 days, from 1 day to 5 days, or 1 day to 7 days.

### Flow Cytometric Assay

Following the combining, the methods may include flow cytometrically assaying the assay composition. By “flow cytometrically assaying” is meant performing a flow cytometric assay on a composition, e.g., an assay composition as described above. The flow cytometric assaying may include characterizing a sample, e.g., a sample including the assay composition, with a flow cytometer system. The flow cytometric assaying may include introducing the assay composition into a flow cytometer. A flow cytometer typically includes a sample reservoir for receiving a fluid sample, such as a sample including the assay composition, and a sheath reservoir containing a sheath fluid. The flow cytometer transports the particles (including cells, e.g., from the assay composition) in the fluid sample as a cell stream to a flow cell, while also directing the sheath fluid to the flow cell. To characterize the components of the flow stream, the flow stream is irradiated with light. Variations in the materials in the flow stream, such as morphologies or the presence of fluorescent labels, may cause variations in the observed light and these variations allow for characterization and separation. For example, particles, such as molecules, analyte-bound beads, or individual cells, in a fluid suspension are passed by a detection region in which the particles are exposed to an excitation light, typically from one or more lasers, and the light scattering and fluorescence properties of the particles are measured. Particles or components thereof typically are labeled with fluorescent dyes to facilitate detection. A multiplicity of different particles or components may be simultaneously detected by using spectrally distinct fluorescent dyes to label the different particles or components. In some implementations, a multiplicity of detectors, one for each of the scatter parameters to be measured, and one or more for each of the distinct dyes to be detected are included in the analyzer. For example, some embodiments include spectral configurations where more than one sensor or detector is used per dye. The data obtained include the signals measured for each of the light scatter detectors and the fluorescence emissions. In certain embodiments, the flow cytometric assay may detect a signal indicating the presence of the labeled secondary antibody in the sample. Where a signal is detected, the sample may include an antibody (antibodies) to the antigenic determinant of the coronaviral antigen.

As summarized above, a sample (e.g., in a flow stream of the flow cytometer) may be irradiated with light from a light source. In some embodiments, the light source is a broadband light source, emitting light having a broad range of wavelengths, such as for example, spanning 50 nm or more, such as 100 nm or more, such as 150 nm or more, such as 200 nm or more, such as 250 nm or more, such as 300 nm or more, such as 350 nm or more, such as 400 nm or more and including spanning 500 nm or more. For example, one suitable broadband light source emits light having wavelengths from 200 nm to 1500 nm. Another example of a suitable broadband light source includes a light source that emits light having wavelengths from 400 nm to 1000 nm. Where methods include irradiating with a broadband light source, broadband light source protocols of interest may include, but are not limited to, a halogen lamp, deuterium arc lamp, xenon arc lamp, stabilized fiber-coupled broadband light source, a broadband LED with continuous spectrum, superluminescent emitting diode, semiconductor light emitting diode, wide spectrum LED white light source, an multi-LED integrated white light source, among other broadband light sources or any combination thereof.

In other embodiments, methods includes irradiating with a narrow band light source emitting a particular wavelength or a narrow range of wavelengths, such as for example with a light source which emits light in a narrow range of wavelengths like a range of 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light (i.e., monochromatic light). Where methods include irradiating with a narrow band light source, narrow band light source protocols of interest may include, but are not limited to, a narrow wavelength LED, laser diode or a broadband light source coupled to one or more optical bandpass filters, diffraction gratings, monochromators or any combination thereof.

In certain embodiments, methods include irradiating the sample with one or more lasers. As discussed above, the type and number of lasers will vary depending on the sample as well as desired light collected and may be a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCl) excimer laser or xenon-fluorine (XeF) excimer laser or a combination thereof. In others instances, the methods include irradiating the flow stream with a dye laser, such as a stilbene, coumarin or rhodamine laser. In yet other instances, methods include irradiating the flow stream with a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, methods include irradiating the flow stream with a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulim YAG laser, ytterbium YAG laser, ytterbium₂O₃ laser or cerium doped lasers and combinations thereof.

The sample may be irradiated with one or more of the above mentioned light sources, such as 2 or more light sources, such as 3 or more light sources, such as 4 or more light sources, such as 5 or more light sources and including 10 or more light sources. The light source may include any combination of types of light sources. For example, in some embodiments, the methods include irradiating the sample in the flow stream with an array of lasers, such as an array having one or more gas lasers, one or more dye lasers and one or more solid-state lasers. In certain instances, the flow stream is irradiated with a plurality of beams of frequency-shifted light and a cell in the flow stream is imaged by fluorescence imaging using radiofrequency tagged emission (FIRE) to generate a frequency-encoded image, such as those described in Diebold, et al. Nature Photonics Vol. 7(10); 806-810 (2013) as well as described in U.S. Pat. Nos. 9,423,353; 9,784,661 and 10,006,852 and U.S. Patent Publication Nos. 2017/0133857 and 2017/0350803, the disclosures of which are herein incorporated by reference.

Aspects of the present methods include collecting fluorescent light with a fluorescent light detector. A fluorescent light detector may, in some instances, be configured to detect fluorescence emissions from fluorescent molecules, e.g., labeled specific binding members (such as labeled antibodies that specifically bind to markers of interest) associated with the particle in the flow cell. In certain embodiments, methods include detecting fluorescence from the sample with one or more fluorescent light detectors, such as 2 or more, such as 3 or more, such as 4 or more, such as 5 or more, such as 6 or more, such as 7 or more, such as 8 or more, such as 9 or more, such as 10 or more, such as 15 or more and including 25 or more fluorescent light detectors. In embodiments, each of the fluorescent light detectors is configured to generate a fluorescence data signal. Fluorescence from the sample may be detected by each fluorescent light detector, independently, over one or more of the wavelength ranges of 200 nm-1200 nm. In some instances, methods include detecting fluorescence from the sample over a range of wavelengths, such as from 200 nm to 1200 nm, such as from 300 nm to 1100 nm, such as from 400 nm to 1000 nm, such as from 500 nm to 900 nm and including from 600 nm to 800 nm. In other instances, methods include detecting fluorescence with each fluorescence detector at one or more specific wavelengths. For example, the fluorescence may be detected at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof, depending on the number of different fluorescent light detectors in the subject light detection system. In certain embodiments, methods include detecting wavelengths of light which correspond to the fluorescence peak wavelength of certain fluorophores present in the sample. In embodiments, fluorescent flow cytometer data is received from one or more fluorescent light detectors (e.g., one or more detection channels), such as 2 or more, such as 3 or more, such as 4 or more, such as 5 or more, such as 6 or more and including 8 or more fluorescent light detectors (e.g., 8 or more detection channels).

Light from the sample may be measured at one or more wavelengths of, such as at 5 or more different wavelengths, such as at 10 or more different wavelengths, such as at 25 or more different wavelengths, such as at 50 or more different wavelengths, such as at 100 or more different wavelengths, such as at 200 or more different wavelengths, such as at 300 or more different wavelengths and including measuring the collected light at 400 or more different wavelengths.

In certain embodiments, methods include spectrally resolving the light from each fluorophore of the fluorophore-biomolecule reagent pairs in the sample. In some embodiments, the overlap between each different fluorophore is determined and the contribution of each fluorophore to the overlapping fluorescence is calculated. In some embodiments, spectrally resolving light from each fluorophore includes calculating a spectral unmixing matrix for the fluorescence spectra for each of the plurality of fluorophores having overlapping fluorescence in the sample detected by the light detection system. In certain instances, spectrally resolving the light from each fluorophore and calculating a spectral unmixing matrix for each fluorophore may be used to estimate the abundance of each fluorophore, such as for example to resolve the abundance of target cells in the sample.

In certain embodiments, methods include spectrally resolving light detected by a plurality of photodetectors such as described e.g., in International Patent Application No. PCT/US2019/068395 filed on Dec. 23, 2019; U.S. Provisional Patent Application No. 62/971,840 filed on Feb. 7, 2020 and U.S. Provisional Patent Application No. 63/010,890 filed on Apr. 16, 2020, the disclosures of which are herein incorporated by reference in their entirety. For example, spectrally resolving light detected by the plurality of photodetectors of the second set of photodetectors may be include solving a spectral unmixing matrix using one or more of: 1) a weighted least square algorithm; 2) a Sherman-Morrison iterative inverse updater; 3) an LU matrix decomposition, such as where a matrix is decomposed into a product of a lower-triangular (L) matrix and an upper-triangular (U) matrix; 4) a modified Cholesky decomposition; 5) by QR factorization; and 6) calculating a weighted least squares algorithm by singular value decomposition.

In certain embodiments, methods further include characterizing the spillover spreading of the light detected by a plurality of photodetectors such as described e.g., in U.S. Provisional Patent Application No. 63/076,611, the disclosure of which is herein incorporated by reference.

In certain instances, the abundance of fluorophores associated with (e.g., chemically associated (i.e., covalently, ionically) or physically associated) a target particle is calculated from the spectrally resolved light from each fluorophore associated with the particle. For instance, in one example the relative abundance of each fluorophore associated with a target particle is calculated from the spectrally resolved light from each fluorophore. In another example, the absolute abundance of each fluorophore associated with the target particle is calculated from the spectrally resolved light from each fluorophore. In certain embodiments, a particle may be identified or classified based on the relative abundance of each fluorophore determined to be associated with the particle. In these embodiments, the particle may be identified or classified by any convenient protocol such as by: comparing the relative or absolute abundance of each fluorophore associated with a particle with a control sample having particles of known identity; or by conducting spectroscopic or other assay analysis of a population of particles (e.g., cells) having the calculated relative or absolute abundance of associated fluorophores.

In certain embodiments, methods include sorting one or more of the particles (e.g., cells) of the sample that are identified based on the estimated abundance of the fluorophores associated with the particle. The term “sorting” is used herein in its conventional sense to refer to separating components (e.g., droplets containing cells, droplets containing non-cellular particles such as biological macromolecules) of a sample and in some instances, delivering the separated components to one or more sample collection containers. For example, methods may include sorting 2 or more components of the sample, such as 3 or more components, such as 4 or more components, such as 5 or more components, such as 10 or more components, such as 15 or more components and including sorting 25 or more components of the sample.

In sorting particles identified based on the abundance of fluorophores associated with the particle, methods include data acquisition, analysis and recording, such as with a computer, where multiple data channels record data from each detector used in obtaining the overlapping spectra of the plurality of fluorophore-biomolecule reagent pairs associated with the particle. In these embodiments, analysis includes spectrally resolving light (e.g., by calculating the spectral unmixing matrix) from the plurality of fluorophores of the fluorophore-biomolecule reagent pairs having overlapping spectra that are associated with the particle and identifying the particle based on the estimated abundance of each fluorophore associated with the particle. This analysis may be conveyed to a sorting system which is configured to generate a set of digitized parameters based on the particle classification.

In some embodiments, methods for sorting components of a sample include sorting particles (e.g., cells in a biological sample), such as described in U.S. Pat. Nos. 3,960,449; 4,347,935; 4,667,830; 5,245,318; 5,464,581; 5,483,469; 5,602,039; ,643,796; 5,700,692; 6,372,506 and 6,809,804, the disclosures of which are herein incorporated by reference. In some embodiments, methods include sorting components of the sample with a particle sorting module, such as those described in U.S. Pat. Nos. 9,551,643 and 10,324,019, U.S. Patent Publication No. 2017/0299493 and International Patent Publication No. WO/2017/040151, the disclosure of which is incorporated herein by reference. In certain embodiments, cells of the sample are sorted using a sort decision module having a plurality of sort decision units, such as those described in U.S. patent application Ser. No. 16/725,756, filed on Dec. 23, 2019, the disclosure of which is incorporated herein by reference.

Flow cytometric assay procedures are well known in the art. See, e.g., Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain aspects, flow cytometrically assaying the composition involves using a flow cytometer capable of simultaneous excitation and detection of multiple fluorophores, such as a BD Biosciences FACSCanto™ flow cytometer, used substantially according to the manufacturer's instructions. Methods of the present disclosure may involve image cytometry, such as is described in Holden et al. (2005) Nature Methods 2:773 and Valet, et al. 2004 Cytometry 59:167-171, the disclosures of which are incorporated herein by reference.

As discussed above, the method includes cytometric analysis which may include sorting. Cells of interest identified in the sample may be sorted and subsequently analyzed by any convenient analysis technique. Subsequent analysis techniques of interest include, but are not limited to, sequencing; assaying by CellSearch, as described in Food and Drug Administration (2004) Final rule. Fed Regist 69: 26036-26038; assaying by CTC Chip, as described in Nagrath, et al. (2007) Nature 450: 1235-1239; assaying by MagSweeper, as described in Talasaz, et al. (2009). Proc Natl Acad Sci U S A 106: 3970-3975; and assaying by nanostructured substrates, as described in Wang S, et al. (2011) Angew Chem Int Ed Engl 50: 3084-3088; the disclosures of which are incorporated herein by reference. Where desired, the sorting protocol may include distinguishing viable and dead cells, where any convenient staining protocol for identifying such cells may be incorporated in to the methods.

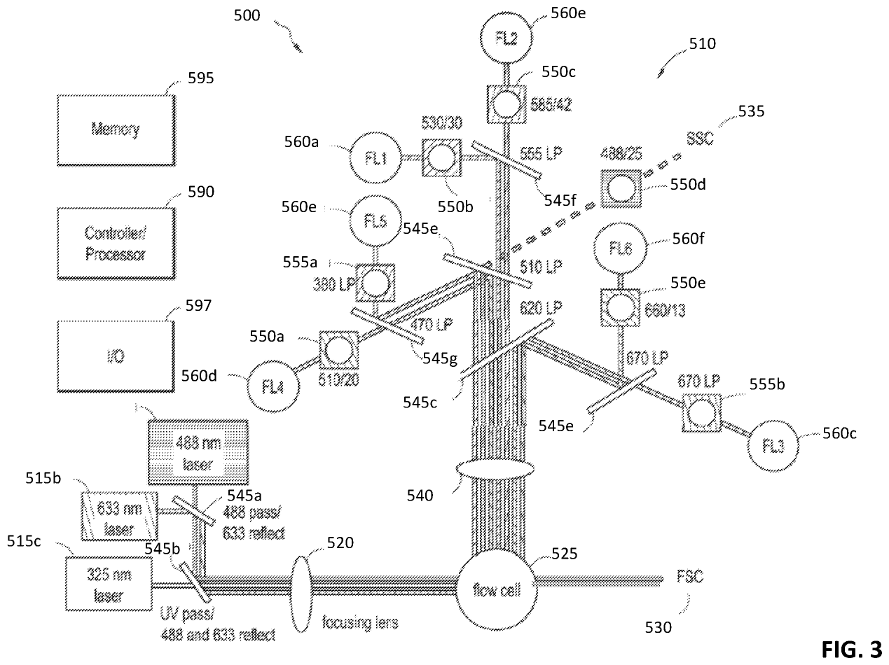

FIG. 3 shows a system 500 for flow cytometry in accordance with an illustrative embodiment of the present invention. The system 500 includes a flow cytometer 510, a controller/processor 590 and a memory 595. The flow cytometer 510 includes one or more excitation lasers 515a-515c, a focusing lens 520, a flow chamber 525, a forward scatter detector 530, a side scatter detector 535, a fluorescence collection lens 540, one or more beam splitters 545a-545g, one or more bandpass filters 550a-550e, one or more longpass (“LP”) filters 555a-555b, and one or more fluorescent detectors 560a-560f.

The excitation lasers 515a-c emit light in the form of a laser beam. The wavelengths of the laser beams emitted from excitation lasers 515a-515c may be 488 nm, 633 nm, and 325 nm, respectively. The laser beams are first directed through one or more of beam splitters 545a and 545b. Beam splitter 445a transmits light at 488 nm and reflects light at 633 nm. Beam splitter 545b transmits UV light (light with a wavelength in the range of 10 to 400 nm) and reflects light at 488 nm and 633 nm.

The laser beams are then directed to a focusing lens 520, which focuses the beams onto the portion of a fluid stream where particles of a sample are located, within the flow chamber 525. The flow chamber is part of a fluidics system which directs particles, typically one at a time, in a stream to the focused laser beam for interrogation. The flow chamber can comprise a flow cell in a benchtop cytometer or a nozzle tip in a stream-in-air cytometer.

The light from the laser beam(s) interacts with the particles in the sample by diffraction, refraction, reflection, scattering, and absorption with re-emission at various different wavelengths depending on the characteristics of the particle such as its size, internal structure, and the presence of one or more fluorescent molecules attached to or naturally present on or in the particle. The fluorescence emissions as well as the diffracted light, refracted light, reflected light, and scattered light may be routed to one or more of the forward scatter detector 530, the side scatter detector 535, and the one or more fluorescent detectors 560a-560f through one or more of the beam splitters 545a-545g, the bandpass filters 550a-550e, the longpass filters 555a-555b, and the fluorescence collection lens 540.

The fluorescence collection lens 540 collects light emitted from the particle- laser beam interaction and routes that light towards one or more beam splitters and filters. Bandpass filters, such as bandpass filters 550a-550e, allow a narrow range of wavelengths to pass through the filter. For example, bandpass filter 550a is a 510/20 filter. The first number represents the center of a spectral band. The second number provides a range of the spectral band. Thus, a 510/20 filter extends 10 nm on each side of the center of the spectral band, or from 500 nm to 520 nm. Shortpass filters transmit wavelengths of light equal to or shorter than a specified wavelength. Longpass filters, such as longpass filters 555a-555b, transmit wavelengths of light equal to or longer than a specified wavelength of light. For example, longpass filter 555a, which is a 670 nm longpass filter, transmits light equal to or longer than 670 nm. Filters are often selected to optimize the specificity of a detector for a particular fluorescent dye. The filters can be configured so that the spectral band of light transmitted to the detector is close to the emission peak of a fluorescent dye.

Beam splitters direct light of different wavelengths in different directions. Beam splitters can be characterized by filter properties such as shortpass and longpass. For example, beam splitter 545g is a 620 SP beam splitter, meaning that the beam splitter 545g transmits wavelengths of light that are 620 nm or shorter and reflects wavelengths of light that are longer than 620 nm in a different direction. In one embodiment, the beam splitters 545a-545g can comprise optical mirrors, such as dichroic mirrors.

The forward scatter detector 530 is positioned slightly off axis from the direct beam through the flow cell and is configured to detect diffracted light, the excitation light that travels through or around the particle in mostly a forward direction. The intensity of the light detected by the forward scatter detector is dependent on the overall size of the particle. The forward scatter detector can include a photodiode. The side scatter detector 535 is configured to detect refracted and reflected light from the surfaces and internal structures of the particle, and tends to increase with increasing particle complexity of structure. The fluorescence emissions from fluorescent molecules associated with the particle can be detected by the one or more fluorescent detectors 560a-560f. The side scatter detector 535 and fluorescent detectors can include photomultiplier tubes. The signals detected at the forward scatter detector 530, the side scatter detector 535 and the fluorescent detectors can be converted to electronic signals (voltages) by the detectors. This data can provide information about the sample.

One of skill in the art will recognize that a flow cytometer in accordance with an embodiment of the present invention is not limited to the flow cytometer depicted in FIG. 3, but can include any flow cytometer known in the art. For example, a flow cytometer may have any number of lasers, beam splitters, filters, and detectors at various wavelengths and in various different configurations.

In operation, cytometer operation is controlled by a controller/processor 590, and the measurement data from the detectors can be stored in the memory 595 and processed by the controller/processor 590. Although not shown explicitly, the controller/processor 590 is coupled to the detectors to receive the output signals therefrom, and may also be coupled to electrical and electromechanical components of the flow cytometer 500 to control the lasers, fluid flow parameters, and the like. Input/output (I/O) capabilities 597 may be provided also in the system. The memory 595, controller/processor 590, and I/O 597 may be entirely provided as an integral part of the flow cytometer 510. In such an embodiment, a display may also form part of the I/O capabilities 597 for presenting experimental data to users of the cytometer 500. Alternatively, some or all of the memory 595 and controller/processor 590 and I/O capabilities may be part of one or more external devices such as a general purpose computer. In some embodiments, some or all of the memory 595 and controller/processor 590 can be in wireless or wired communication with the cytometer 510. The controller/processor 590 in conjunction with the memory 595 and the I/O 597 can be configured to perform various functions related to the preparation and analysis of a flow cytometer experiment.

The system illustrated in FIG. 3 includes six different detectors that detect fluorescent light in six different wavelength bands (which may be referred to herein as a “filter window” for a given detector) as defined by the configuration of filters and/or splitters in the beam path from the flow cell 525 to each detector. Different fluorescent molecules used for a flow cytometer experiment will emit light in their own characteristic wavelength bands. The particular fluorescent labels used for an experiment and their associated fluorescent emission bands may be selected to generally coincide with the filter windows of the detectors. However, as more detectors are provided, and more labels are utilized, perfect correspondence between filter windows and fluorescent emission spectra is not possible. It is generally true that although the peak of the emission spectra of a particular fluorescent molecule may lie within the filter window of one particular detector, some of the emission spectra of that label will also overlap the filter windows of one or more other detectors. This may be referred to as spillover. The I/O 597 can be configured to receive data regarding a flow cytometer experiment having a panel of fluorescent labels and a plurality of cell populations having a plurality of markers, each cell population having a subset of the plurality of markers. The I/O 597 can also be configured to receive biological data assigning one or more markers to one or more cell populations, marker density data, emission spectrum data, data assigning labels to one or more markers, and cytometer configuration data. Flow cytometer experiment data, such as label spectral characteristics and flow cytometer configuration data can also be stored in the memory 595. The controller/processor 590 can be configured to evaluate one or more assignments of labels to markers.

### Specific Embodiment

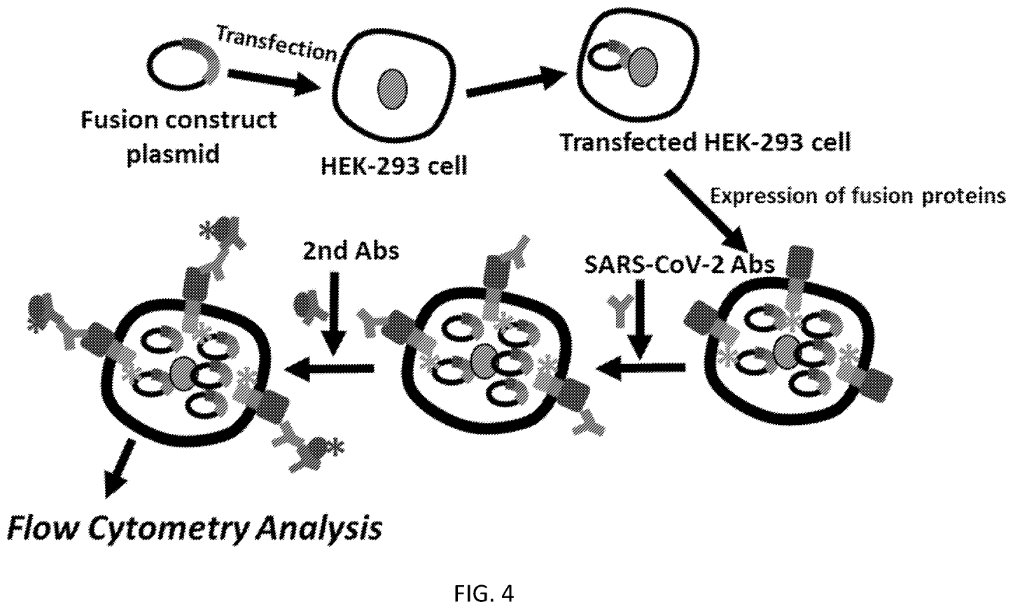

FIG. 4 provides a schematic representation of an embodiment of a cell-based serological assay for antibodies specific to a SARS-CoV-2 antigen. In FIG. 4, an HEK-293 cell used to detect antibodies in a sample is produced and then incubated with a sample before being subjected to flow cytometric analysis. To produce an HEK-293 cell expressing an epitope of a SARS-CoV-2 antigen, an HEK-293 cell is transfected with a fusion construct plasmid under conditions suitable for transfection. The fusion construct plasmid encodes a fusion protein that includes an epitope for a SARS-CoV-2 antigen. The transfected HEK-293 cell is cultured under conditions where the cell expresses the fusion protein. The expressed fusion protein includes the SARS-CoV-2 epitope, a transmembrane domain, and a green fluorescent protein. After expression, the fusion protein will be translocated and incorporated into the cell membrane with an anchoring domain derived from a transmembrane domain of another protein. The fusion protein is further oriented such that the SARS-CoV-2 protein is positioned on the surface of the cell membrane and the fluorescent protein is positioned inside the cell on the cytoplasmic side of the cell membrane. The HEK-293 cell expressing the fusion protein is then incubated with a sample from a subject such as a saliva, serum, or plasma sample. If the sample contains antibodies specific to the SARS-CoV-2 epitope present on the surface of the HEK-293 cell, then the antibodies will bind to the epitope expressed on the cell. The sample containing the cell with bound antibodies is then incubated with fluorescently labeled secondary antibodies, e.g., PE or APC mouse anti-human antibodies, that are specific to the antibodies bound to the SARS-COV-2 epitope. After incubating the sample with the secondary antibodies, the sample is introduced to a flow cytometer. The flow cytometer detects a fluorescent signal from the cell if secondary antibodies are bound to the cell, i.e., bound to antibodies from the subject that are bound to the cell. The presence of the signal indicates the sample obtained from the patient included antibodies specific to a SARS-CoV-2 antigen. As such, the recombinant fusion proteins expressed on the cell will serve as antigens for capturing a patient's antibodies to SARS-CoV-2. The presence of the signal further indicates the subject has a had an immune response to and/or may be immune to a future infection by SARS-CoV-2.

### Additional Aspects

In some cases, the method further includes determining an amount, e.g., titer, of the antibody that binds to the coronaviral antigen that is present in the sample. In some cases, the determining includes serially diluting the sample. A serial dilution may include a step-wise and geometric series of dilutions. To make a single dilution, a known volume of starting material, e.g., sample, may be combined with a known volume of diluent to produce a dilute solution. For example, 1 ml of sample may be combined with 9 ml of diluent to produce a 10-fold dilution. The process for making a single dilution is repeated sequentially using the previously prepared dilute solution as the starting material. For example, 1 ml of the previous dilute solution may be added to 9 ml of diluent in the next step of dilution, and each subsequent step would result in a further 10-fold change in the concentration from the previous concentration. To calculate antibody titer, a sample containing the antibody may be serially diluted and each dilution may be tested for the presence of detectable levels of the antibody. The determined titer is indicative of the last dilution in which the antibody was detected. The serial dilution may occur at any time in the above disclosed methods including, e.g., before combining the sample with the mammalian cell and labeled secondary binding member. In certain embodiments, the sample is a sample from a patient that has recovered from a coronaviral infection. In such embodiments, the patient may be an individual that has previously had a coronaviral infection, e.g., a COVID-19 infection, and no longer shows symptoms of infection or has cleared the virus from the body (e.g., as shown by a negative RT-PCR-based test for the virus).

### Analysis of Flow Cytometric Assay Results

Aspects of the methods may further include methods of assessing a subject for an immune response to a coronaviral infection based on the results obtained from flow cytometrically assaying the assay composition. By “assessing a subject for an immune response” is meant assaying a sample from a subject for a target antibody according to any of the embodiments described herein and determining whether the subject has or has had an immune response to a coronavirus based on whether the target antibody is present in the sample. The presence of the target antibody in the sample may indicate that the subject has or has had an immune response to a coronavirus. The presence of the target antibody in the sample may indicate that the subject has or has had a coronaviral infection. The absence of the target antibody in the sample may indicate that the subject has not had an immune response to a coronavirus, e.g., an antibody generating immune response. The absence of the target antibody in the sample may indicate that the subject has not had a coronaviral infection. The presence of the target antibody may be indicated by the presence of a signal from a labeled secondary binding member that binds to the target antibody, as described above. The presence of a signal from the labeled secondary binding member may indicate that the sample includes the antibody and that the subject has had an immune response to a coronavirus. The signal may be obtained, e.g., detected, flow cytometrically as described above. In some cases, the presence of a signal from the labeled secondary binding member indicates that the subject has produced antibodies to the coronavirus and has immunity to the coronavirus.

In practicing embodiments of the methods, aspects of the method may include combining, according to any of the embodiments disclosed herein, a sample from the subject, a mammalian cell displaying on a surface thereof an antigenic determinant of a coronaviral antigen that is specifically bound by an antibody that may be present in the sample, and a labeled secondary binding member that binds to the antibody to produce an assay composition; and flow cytometrically assaying the assay composition, according to any of the embodiments disclosed herein, for the presence of a signal from the labeled secondary binding member to determine whether the sample includes the antibody and whether the subject has had an immune response to a coronaviral infection.

The sample may be assayed at any point during a subject's infection with a coronavirus. In certain embodiments, the subject has, has had, is suspected to have, or is suspected to have had a coronaviral infection. As used herein, a “coronaviral infection” refers to an infection of a subject by a coronavirus including, e.g., SARS-CoV, MERS-CoV, or SARS-CoV-2. A subject with a coronaviral infection may exhibit one or more symptoms including, e.g., a cough, fever or chills, shortness of breath, fatigue, muscle or body aches, new loss of taste or smell, sore throat, headache, congestion, nausea, vomiting, diarrhea, chest pain or pressure, confusion, inability to wake or stay awake, and bluish lips or face. In some cases, the subject is asymptomatic. In some cases, a subject with a coronaviral infection exhibits one or more syndromes or acute conditions including, e.g., organ failure, acute respiratory distress syndrome, acute kidney injury, and thrombosis. In certain embodiments, the patient has or is expected to develop symptoms associated with a cytokine response, e.g., a cytokine storm caused by the overproduction of inflammatory cytokines. In some cases, the coronaviral infection is a SARS infection. In some cases, the coronaviral infection is a MERS infection. In some cases, the coronaviral infection is a COVID-19 infection.

### Systems

Aspects of the present disclosure also include systems suitable for practicing embodiments of the methods described herein. The systems may determine, e.g., may be configured to determine, whether a sample includes a target antibody that binds to an antigenic determinant of a coronaviral antigen. The systems may be configured to flow cytometrically assay an assay composition including a mammalian cell, a labeled secondary binding member and a sample from a subject. In some cases, the system detects a signal from the labeled secondary binding member when the sample includes a target antibody that binds to an antigenic determinant of a coronaviral antigen. For example, the system may detect a fluorescence signal from a labeled secondary binding member that is bound to an antibody in the sample that specifically binds to the antigenic determinant present on the surface of the mammalian cell.

Systems for use in practicing the subject methods may include a mammalian cell displaying on a surface thereof an antigenic determinant of a coronaviral antigen, a labeled secondary binding member that binds to an antibody that specifically binds to the antigenic determinant, and a flow cytometric system configured to assay an assay composition including the mammalian cell, the labeled secondary binding member, and the sample for the presence of a signal from the labeled secondary binding member to determine whether the sample includes the antibody.

The flow cytometric system may be configured to perform a flow cytometric assay, e.g., may include any convenient flow cytometer, according to any of the embodiments described herein. In some cases, the systems include a flow type particle sorting system. Flow-type particle sorting systems, such as sorting flow cytometers, are used to sort particles in a fluid sample based on at least one measured characteristic of the particles. In a flow-type particle sorting system, particles, such as molecules, analyte-bound beads, or individual cells, in a fluid suspension are passed in a stream by a detection region in which a sensor detects particles contained in the stream of the type to be sorted. The sensor, upon detecting a particle of the type to be sorted, triggers a sorting mechanism that selectively isolates the particle of interest.

Particle sensing typically is carried out by passing the fluid stream by a detection region in which the particles are exposed to irradiating light, from one or more lasers, and the light scattering and fluorescence properties of the particles are measured. Particles or components thereof can be labeled with fluorescent dyes to facilitate detection, and a multiplicity of different particles or components may be simultaneously detected by using spectrally distinct fluorescent dyes to label the different particles or components. Detection is carried out using one or more photosensors to facilitate the independent measurement of the fluorescence of each distinct fluorescent dye.

One type of flow-type particle sorting system is the electrostatic sorting type. In an electrostatic sorter, a fluid suspension is jetted from a nozzle and vibrated to break the stream into uniform discrete drops. The sorting mechanism includes a drop charging means connected to the stream to charge drops containing a particle of the type to be sorted with an electrical charge as it breaks off from the jet stream. The stream of drops is passed through a transverse electrostatic field established by a pair of oppositely charged deflection plates. Charged drops containing a particle of the type to be sorted are deflected in a direction and in an amount related to the polarity and magnitude of the drop charge and are collected in distinct collection receptacles. Uncharged drops are not deflected passing through the electrostatic field and are collected by a central receptacle.

Where flow cytometric analysis is performed, any convenient flow cytometry system may be employed. In certain instances, flow cytometry systems of interest include BD Biosciences BD Biosciences FACSCanto™ II flow cytometer, BD Accuri™ flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer BD Biosciences LSRFortessa™ flow cytometer, BD Biosciences LSRFortess™ X-20 flow cytometer and BD Biosciences FACSCalibur™ cell sorter, a BD Biosciences FACSCount™ cell sorter, BD Biosciences FACSLyric™ cell sorter and BD Biosciences Via™ cell sorter BD Biosciences Influx™ cell sorter, BD Biosciences Jazz™ cell sorter, BD Biosciences Aria™ cell sorters and BD Biosciences FACSMelody™ cell sorter, or the like. In certain embodiments, the subject systems are flow cytometric systems, such those described in U.S. Pat. Nos. 3,960,449; 4,347,935; 4,667,830; 4,704,891; 4,770,992; 5,030,002; 5,040,890; 5,047,321; 5,245,318; 5,317,162; 5,464,581; 5,483,469; 5,602,039; 5,620,842; 5,627,040; 5,643,796; 5,700,692; 6,372,506;6,809,804; 6,813,017; 6,821,740; 7,129,505; 7,201,875; 7,544,326; 8,140,300; 8,233,146; 8,753,573; 8,975,595; 9,092,034; 9,095,494 and 9,097,640; the disclosure of which are herein incorporated by reference in their entirety.

In certain embodiments, the subject flow cytometric systems are configured to sort one or more of the particles (e.g., cells) of the sample. For example, the subject systems may be configured for sorting samples having 2 or more components, such as 3 or more components, such as 4 or more components, such as 5 or more components, such as 10 or more components, such as 15 or more components and including soring a sample having 25 or more components. One or more of the sample components may be separated from the sample and delivered to a sample collection container, such as 2 or more sample components, such as 3 or more sample components, such as 4 or more sample components, such as 5 or more sample components, such as 10 or more sample components and including 15 or more sample components may be separated from the sample and delivered to a sample collection container.

In some embodiments, particle sorting systems of interest are configured to sort particles with an enclosed particle sorting module, such as those described in U.S. Patent Publication No. 2017/0299493, filed on Mar. 28, 2017, the disclosure of which is incorporated herein by reference. In certain embodiments, particles (e.g., cells) of the sample are sorted using a sort decision module having a plurality of sort decision units, such as those described in U.S. Patent Application No. 16/725,756, filed on Dec. 23, 2019, the disclosure of which is incorporated herein by reference. In some embodiments, the subject particle sorting systems are flow cytometric systems, such those described in U.S. Pat. Nos. 10,006,852; 9,952,076; 9,933,341; 9,784,661; 9,726,527; 9,453,789; 9,200,334; 9,097,640; 9,095,494; 9,092,034; 8,975,595; 8,753,573; 8,233,146; 8,140,300; 7,544,326; 7,201,875; 7,129,505; 6,821,740; 6,813,017; 6,809,804; 6,372,506; 5,700,692; 5,643,796; 5,627,040; 5,620,842; 5,602,039; the disclosure of which are herein incorporated by reference in their entirety.

### Utility

The subject methods, systems, expression vectors, cells, and kits find use in clinical and research applications involving antibodies directed to a viral antigen. Suitable applications include those where it is desirable to determine the immune response status of an individual, e.g., whether the individual has had an immune response to a viral infection. The subject methods, systems, expression vectors, cells, and kits may find use in determining if a subject has had an immune response to a coronavirus and/or has had a coronaviral infection, e.g., a SARS infection, a MERS infection, or a COVID-19 infection. The subject methods, systems, expression vectors, cells, and kits may provide information about how the immune system responds to a coronavirus at different stages of infection. In certain embodiments, the subject methods, systems, expression vectors, cells, and kits may indicate whether an asymptomatic individual has antibodies and/or immunity to a coronavirus. Additional applications include determining whether an antibody specific to a coronaviral antigen is present in a sample from a subject.

Other suitable applications for the subject methods, systems, expression vectors, cells, and kits include determining the titer of antibodies in recovered individuals. In certain embodiments, the subject methods, systems, expression vectors, cells, and kits find use in determining the titer of antibodies in convalescent plasma from a recovered individual for clinical or research purposes. In some cases, the determining of the titer of antibodies in convalescent plasma provides information on the suitable titer of antibodies needed to treat a subject with a coronaviral infection. In some cases, determining the titer of antibodies finds use in treating a coronavirus patient with convalescent plasma, e.g., by determining if the plasma sample has an effective amount of antibody specific to an antigenic determinant of a coronaviral antigen to treat the patient. As used herein in its conventional sense, “convalescent plasma” refers to the plasma from an individual who has recovered from a viral infection, e.g., a coronaviral infection. In some instances, convalescent plasma may be administered to a subject with a coronaviral infection to treat the subject.

### Kits

Aspects of the present disclosure also include kits. The kits may be suitable for practicing any of the subject methods. The kits may be compatible with any of the subject systems. The kits may include, e.g., a mammalian cell displaying on a surface thereof an antigenic determinant of a coronaviral antigen, according to any of the embodiments described herein; and a labeled secondary binding member that binds to an antibody that specifically binds to the antigenic determinant, according to any of the embodiments described herein.

The kit may include any suitable amount of mammalian cells. In some cases, the kit includes an effective amount of mammalian cells for determining if a sample includes an antibody that binds to an antigenic determinant of a coronaviral antigen. The mammalian cells may be present in any suitable container that is compatible with the mammalian cells. By “compatible” is meant that the container is substantially inert (e.g., does not significantly react with) the cells, liquid and/or reagent(s) in contact with a surface of the container. Containers of interest may vary and may include but are not limited to a test tube, centrifuge tube, culture tube, falcon tube, microtube, Eppendorf tube, specimen collection container, specimen transport container, and syringe. In some embodiments, the kits include an amount of cells ranging from 1×10² to 1×10⁶ cells such as, e.g., 1×10² to 1×10⁵ cells.

The kit may include any suitable amount of the labeled secondary binding member. In some cases, the kit includes an effective amount of a labeled secondary binding member for determining if a sample includes an antibody that binds to an antigenic determinant of a coronaviral antigen. In some embodiments, the kit includes an amount of labeled secondary binding member ranging from 1 μg to 500 μg, such as 10 μg to 100 μg and including 10 μg to 50 μg. The labeled secondary binding member may be present in any suitable container that is compatible with the labeled secondary binding member. Containers of interest may vary and may include but are not limited to a test tube, centrifuge tube, culture tube, falcon tube, microtube, Eppendorf tube, specimen collection container, specimen transport container, and syringe.

The container for holding a component of the kit, e.g., the mammalian cell or the labeled secondary binding member, may hold any suitable volume or quantity of the component. In some cases, the size of the container may depend on the quantity or volume of a component to be held in the container. In certain embodiments, the container may be configured to hold an amount of a component ranging from 0.1 mg to 1000 mg, such as from 0.1 mg to 900 mg, such as from 0.1 mg to 800 mg, such as from 0.1 mg to 700 mg, such as from 0.1 mg to 600 mg, such as from 0.1 mg to 500 mg, such as from 0.1 mg to 400 mg, or 0.1 mg to 300 mg, or 0.1 mg to 200 mg, or 0.1 mg to 100 mg, 0.1 mg to 90 mg, or 0.1 mg to 80 mg, or 0.1 mg to 70 mg, or 0.1 mg to 60 mg, or 0.1 mg to 50 mg, or 0.1 mg to 40 mg, or 0.1 mg to 30 mg, or 0.1 mg to 25 mg, or 0.1 mg to 20 mg, or 0.1 mg to 15 mg, or 0.1 mg to 10 mg, or 0.1 mg to 5 mg, or 0.1 mg to 1 mg, or 0.1 mg to 0.5 mg. In certain embodiments, the container is configured to hold an amount of a component ranging from 0.1 g to 10 g, or 0.1 g to 5 g, or 0.1 g to 1 g, or 0.1 g to 0.5 g. In certain instances, the container is configured to hold a volume ranging from 0.1 ml to 200 ml. For instance, the container may be configured to hold a volume (e.g., a volume of a liquid) ranging from 0.1 ml to 1000 ml, such as from 0.1 ml to 900 ml, or 0.1 ml to 800 ml, or 0.1 ml to 700 ml, or 0.1 ml to 600 ml, or 0.1 ml to 500 ml, or 0.1 ml to 400 ml, or 0.1 ml to 300 ml, or 0.1 ml to 200 ml, or 0.1 ml to 100 ml, or 0.1 ml to 50 ml, or 0.1 ml to 25 ml, or 0.1 ml to 10 ml, or 0.1 ml to 5 ml, or 0.1 ml to 1 ml, or 0.1 ml to 0.5ml.

The shape of the container may also vary. In certain cases, the container may be configured in a shape that is compatible with the assay and/or the method or other devices used to perform the assay. For instance, the container may be configured in a shape of typical laboratory equipment used to perform the assay or in a shape that is compatible with other devices used to perform the assay. In some embodiments, the liquid container may be a vial or a test tube. In certain cases, the liquid container is a vial. In certain cases, the liquid container is a test tube.

Examples of suitable materials for the containers include, but are not limited to, glass and plastic. For example, the container may be composed of glass, such as, but not limited to, silicate glass, borosilicate glass, sodium borosilicate glass (e.g., PYREX™), fused quartz glass, fused silica glass, and the like. Other examples of suitable materials for the containers include plastics, such as, but not limited to, polypropylene, polymethylpentene, polytetrafluoroethylene (PTFE), perfluoroethers (PFE), fluorinated ethylene propylene (FEP), perfluoroalkoxy alkanes (PFA), polyethylene terephthalate (PET), polyethylene (PE), polyetheretherketone (PEEK), and the like.

In some embodiments, the container may be sealed. That is, the container may include a seal that substantially prevents the contents of the container from exiting the container. The seal of the container may also substantially prevent other substances from entering the container. For example, the seal may be a water-tight seal that substantially prevents liquids from entering or exiting the container, or may be an air-tight seal that substantially prevents gases from entering or exiting the container. In some instances, the seal is a removable or breakable seal, such that the contents of the container may be exposed to the surrounding environment when so desired, e.g., if it is desired to remove a portion of the contents of the container. In some instances, the seal is made of a resilient material to provide a barrier (e.g., a water-tight and/or air-tight seal) for retaining a sample in the container. Particular types of seals include, but are not limited to, films, such as polymer films, caps, etc., depending on the type of container. Suitable materials for the seal include, for example, rubber or polymer seals, such as, but not limited to, silicone rubber, natural rubber, styrene butadiene rubber, ethylene-propylene copolymers, polychloroprene, polyacrylate, polybutadiene, polyurethane, styrene butadiene, and the like, and combinations thereof. For example, in certain embodiments, the seal is a septum pierceable by a needle, syringe, or cannula. The seal may also provide convenient access to a sample in the container, as well as a protective barrier that overlies the opening of the container. In some instances, the seal is a removable seal, such as a threaded or snap-on cap or other suitable sealing element that can be applied to the opening of the container. For instance, a threaded cap can be screwed over the opening before or after a sample has been added to the container.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed.

(Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: Binding of SARS-CoV-2 Spike Protein Specific Antibodies to HEK293 Cells Containing Spike Protein

HEK293 cells are transfected with a plasmid containing SARS-CoV-2 spike surface glycoprotein. This protein is expressed on the surface of the HEK293 cells as a part of the fusion construct with GFP and transmembrane domain (TMD). The cell line is green fluorescent and binds to SARS-CoV-2 spike antibodies (monoclonal mouse IgG). HEK293 cells expressing just a GFP-TMD fusion protein are used as a control, because this cell line will be green fluorescent but unable to bind SARS-CoV-2 spike antibodies.

Presence of GFP allows to control transfection efficiency of the HEK293 cells and expression of fusion proteins. Serial dilutions of patient plasma containing SARS-CoV-2 spike antibodies are used to incubate with the cell line HEK293 expressing the spike protein and the control HEK293 cells. The excess of the plasma is washed out, followed by 1-2 h incubation at room temperature with PE-labeled anti-mouse IgG secondary reporter antibodies in PBS. This provides a PE signal in proportion to SARS-CoV-2 spike antibodies on the surface of the HEK293 cells. The excess of the PE-labeled anti-mouse IgG secondary antibodies is washed out. The binding capacity for spike antibodies by the HEK293 cells expressing on the surface the spike protein is then assessed using a multi-color flow cytometry.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of determining whether a sample comprises an antibody that binds to a coronaviral antigen, the method comprising:
(a) combining:
(i) the sample;
(ii) a mammalian cell displaying on a surface thereof an antigenic determinant of the coronaviral antigen that is specifically bound by the antibody; and
(iii) a labeled secondary binding member that binds to the antibody to produce an assay composition; and
(b) flow cytometrically assaying the assay composition for the presence of a signal from the labeled secondary binding member to determine whether the sample comprises the antibody.

2. The method of claim 1, wherein the antigenic determinant is a component of a membrane anchored protein.

3. The method of claim 2, wherein the membrane anchored protein comprises a transmembrane protein.

4. The method of claim 2, wherein the membrane anchored protein comprises a lipid anchor.

5. The method of claim 2, wherein the membrane anchored protein comprises a detectible domain.

6. The method of claim 5, wherein the detectible domain is present inside the cell.

7. The method of claim 5, wherein the detectable domain comprises a fluorescent protein.

8. The method of claim 1, wherein the mammalian cell comprises an expression construct encoding the antigenic determinant of the coronaviral antigen.

9-10. (canceled)

11. The method of claim 1, wherein the coronaviral antigen is derived from SARS-CoV.

12. The method of claim 1, wherein the coronaviral antigen is derived from MERS-CoV.

13. The method of claim 1, wherein the coronaviral antigen is derived from SARS-CoV-2.

14. The method of claim 1, wherein the coronaviral antigen comprises a spike protein.

15-19. (canceled)

20. The method of claim 1, wherein the labeled secondary binding member comprises an antibody or binding fragment thereof.

21. (canceled)

22. The method of claim 1, wherein the labeled secondary binding member comprises a fluorescent label.

23-25. (canceled)

26. The method of claim 1, wherein the sample comprises a biological fluid.

27-29. (canceled)

30. The method of claim 1, wherein the method further comprises determining an amount of the antibody that binds to the coronaviral antigen present in the sample.

31-129. (canceled)

130. A kit comprising:
a mammalian cell displaying on a surface thereof an antigenic determinant of a coronaviral antigen; and
a labeled secondary binding member that binds to an antibody that specifically binds to the antigenic determinant.

131. The kit of claim 130, wherein the antigenic determinant is a component of a membrane anchored protein.

132-141. (canceled)

142. The kit of claim 130, wherein the coronaviral antigen is derived from SARS-CoV-2.

143-148. (canceled)

149. The kit of claim 130, wherein the labeled secondary binding member comprises an antibody or binding fragment thereof.

150-165. (canceled)
